(19) United States
(12) Patent Application Publication
(10) Pub. No.: US 2020/0181557 A1
(43) Pub. Date: Jun. 11, 2020

(54) PHOTOBIOREACTOR

(71) Applicant: COMMISSARIAT A L'ENERGIE ATOMIQUE ET AUX ENERGIES ALTERNATIVES, Paris (FR)

(72) Inventor: Cédric DUCROS, Grenoble (FR)

(21) Appl. No.: 16/707,794

(22) Filed: Dec. 9, 2019

(30) Foreign Application Priority Data
Dec. 10, 2018 (FR) 18 72612

(51) Int. Cl. C12M 1/00

(52) U.S. Cl. CPC C12M 31/00; C12M 23/22

(57) ABSTRACT

Photobioreactor for culturing a photosynthetic microorganism, comprising:
an enclosure comprising a light-collecting wall and defining a culture chamber for containing a culture medium containing at least the photosynthetic microorganism, the light-collecting wall being transparent to infrared radiation and to visible radiation, and
an optical filter for filtering irradiation radiation directed toward the culture chamber,
   the optical filter being transparent to radiation in the visible and containing a thermochromic compound,
the optical filter being transparent to infrared radiation at a temperature at least 10° C. below the transition temperature of the thermochromic compound and having an optical transmittance to infrared radiation of 20% or less at a temperature at least 10° C. above the transition temperature of the thermochromic compound.

### TECHNICAL FIELD

The present invention relates to the field of photobioreactors suitable for culturing a photosynthetic microorganism.

### PRIOR ART

A photosynthetic microorganism is usually cultured by means of a photobioreactor. For example, a photobioreactor can be integrated into a building to promote heat exchange between the photobioreactor and the building. A photobioreactor conventionally comprises a tank containing a culture medium containing the photosynthetic microorganism, the cell being generally covered with a light-transparent and more favorably fully transparent cover. Alternatively, the photobioreactor may have a structure consisting of hollow and transparent tubes through which the culture medium circulates, in an open or closed loop. The photobioreactor thus defines a culture chamber that isolates the culture medium and the photosynthetic microorganism from the surrounding atmosphere. The growth of the photosynthetic microorganism is ensured in particular by the exposure of the culture medium to light radiation. For example, when integrated into a building, the photobioreactor is exposed to solar radiation. Such radiation has spectral components in the infrared that cause the culture medium to heat up. In addition, solar radiation varies daily and seasonally, resulting in temperature variations in the growing medium. However, for the growth of the photosynthetic microorganism to be optimal, the temperature of the culture medium must be within a temperature range optimal for the growth of the cultured photosynthetic microorganism, generally between 25° C. and 40° C. However, heating of the culture medium can limit or even block the growth of the photosynthetic microorganism, and if excessive, can cause its death. Excessive mortality of the photosynthetic microorganism leads to degradation of the photobioreactor's performance.

In addition, among the spectrum of solar radiation, the components in the visible are those that promote optimal growth of the photosynthetic microorganism.

To ensure high yield, the culture of the photosynthetic microorganism therefore requires maximizing exposure to light radiation, including the visible components of light radiation, to promote the growth of the photosynthetic microorganism, while ensuring that the temperature of the culture medium is maintained within the optimal growth temperature range specific to the photosynthetic microorganism.

To regulate the temperature of the culture medium, it is known, for example, from WO 2007/129327 A1 to spray the photobioreactor with water, or even to immerse it completely in water. However, such regulation requires an external water supply and the construction of an infrastructure around the photobioreactor, which increases the cost of culturing the photosynthetic microorganism. In addition, spraying or immersion can lead to fouling of the photobioreactor jacket and induce parasitic optical reflections, which reduces the absorption of light radiation by the photosynthetic microorganism.

It is also known to implement a temperature control unit for the culture medium. To this end, application WO 2007/129327 A1 describes an external exchanger through which the culture medium flows. Application FR 2 823 761 A1 describes a photobioreactor with a jacket in which a heat transfer fluid flows to exchange heat with the culture medium. In application EP 0 647 707 A1, the photobioreactor has a wall in which is housed a heating and cooling unit to regulate the temperature of the culture medium. However, the technical solutions described in the three documents mentioned above add to the operating cost of the photobioreactor.

A photobioreactor is also known from patent EP 1 928 994 Bl, which consists of tubes coated with a thermal barrier layer. Application FR 2 964 666 A1 describes a photobioreactor equipped with a thermal valve made of a phase-change material, for example paraffin, so as to passively maintain the temperature of the culture medium below a threshold temperature. Finally, application FR 2 978 772 A1 describes a photobioreactor whose wall is formed by a glazing whose face is covered by a stack of anti-reflective thin layers and functional thin layers. The stack forms an optical filter that transmits only those components of solar radiation that are useful for photosynthesis. The photobioreactor of application FR 2 978 772 A1 thus limits the risk of excessive heating of the culture medium, particularly in summer, as the filter reflects the components in the infrared of solar radiation. However, filtering the components in the infrared can be harmful, especially in winter, because it deprives the culture medium of a beneficial heat supply to maintain the temperature of the culture medium at a minimum value for the growth of the photosynthetic microorganism.

There thus remains a need for a photobioreactor that ensures optimal growth of a photosynthetic microorganism at a lower cost, when exposed to time-varying light radiation, particularly solar radiation.

The invention aims to satisfy this need and proposes a photobioreactor for culturing a photosynthetic microorganism, the photobioreactor comprising:
an enclosure comprising a light-collecting wall and defining a culture chamber for containing a culture medium containing at least the photosynthetic microorganism, the light-collecting wall being transparent to infrared radiation and to visible radiation, and
an optical filter to filter irradiation radiation directed toward the culture chamber, the optical filter being transparent to radiation in the visible and containing at least one thermochromic compound,
   the optical filter being transparent to infrared radiation at a temperature at least 10° C. below the transition temperature of the thermochromic compound and having an optical transmittance to infrared radiation of 20% or less at a temperature at least 10° C. above the transition temperature of the thermochromic compound.

The optical filter of the photobioreactor according to the invention promotes the transmission to the culture chamber of a sufficient portion of the intensity of the spectrum in the visible of the irradiation radiation to ensure optimal photosynthetic activity of the photosynthetic microorganism. In addition, the optical filter passively and cost-effectively regulates the temperature of the culture medium. In particular, when the temperature of the optical filter is below the minimum limit of the optimal temperature range for the growth of the photosynthetic microorganism and is below the transition temperature, for example under winter conditions and/or in early morning and late afternoon, the infrared portion of the irradiation radiation transmitted by the optical filter into the culture chamber promotes the temperature rise of the culture medium. The temperature of the culture medium thus gradually increases until it falls within the optimal growth temperature range of the photosynthetic microorganism. Consequently, the photosynthetic activity of the photosynthetic microorganism becomes more intense. When the temperature of the optical filter is higher than the maximum limit of the optimal temperature range for the growth of the photosynthetic microorganism and higher than the transition temperature, the optical filter limits the transmission of infrared components of irradiation radiation to the culture chamber. The heating of the culture medium is thus limited, and better is nil. Death of the photosynthetic microorganism due to an excessive rise in the temperature of the culture medium can thus be avoided. The temperature of the culture medium then gradually decreases until it falls within the optimal temperature range for the growth of the photosynthetic microorganism. Thus, the photosynthetic activity of the photosynthetic microorganism is intensified.

“Visible radiation” means light radiation whose spectrum comprises at least one, preferably a plurality of components whose respective wavelengths are between 350 nm and 780 nm, preferably between 350 nm and 750 nm.

“Infrared radiation” means light radiation whose spectrum comprises at least one, preferably a plurality of components whose respective wavelengths are greater than 780 nm, preferably greater than 1100 nm.

“Optical transmittance” of a body to radiation refers to the ratio, expressed as a percentage, of the intensity of the flux transmitted through the body to the intensity of the incident radiation flux to which the body is exposed. When the light radiation is polychromatic, the respective intensities of the incident and transmitted fluxes are calculated by integrating, over the wavelength spectrum constituting the light radiation, the elementary fluxes associated with the respective wavelengths. In the sense of the invention, the optical transparency of the body is considered in a temperature range between 0° C. and 70° C.

A body “transparent” to light radiation has an optical transmittance to said light radiation preferably greater than 80% or even greater than 90%.

Irradiation radiation may include components in the infrared and visible regions. It can be solar radiation in particular.

Preferably, the transition temperature of the thermochromic compound is greater than or equal to 30° C., preferably greater than or equal to 35° C. and/or less than or equal to 55° C., and preferably less than or equal to 50° C., or even less than or equal to 45° C. For example, it is between 38° C. and 42° C. This optimizes the performance of the photobioreactor, especially when the photosynthetic activity of the photosynthetic microorganism is optimal in a temperature range between 25° C. and 40° C. Preferably, the transition temperature of the thermochromic compound is chosen according to the nature of the photosynthetic microorganism to be grown.

A “thermochromic” compound is characterized by a reversible variation of an optical property with a variation in the temperature of the compound. The “transition temperature” characterizes a change of state, including a phase change, of the thermochromic compound. The change of state results in a discontinuous variation in the optical indices, i.e. refractive index n and extinction coefficient k, of the thermochromic compound. In particular, the thermochromic compound may have an infrared extinction coefficient at least 3 times higher than the visible extinction coefficient and an infrared refractive index at least 2 times lower than the visible refractive index. In addition, the thermochromic compound preferably has a substantially identical extinction coefficient and/or refractive index in the infrared and in the visible.

The transition temperature can be determined by measuring the optical indices of the thermochromic compound.

For example, measurements are performed using two ellipsometers, each equipped with a heating cell, to heat the thermochromic compound to temperatures between 20° C. and 100° C. Measurements of optical indices in the visible and infrared wavelength ranges are performed using the first and second ellipsometers, respectively, in order to characterize the relationship between the optical indices and the wavelength of the radiation.

The thermochromic compound can be selected from the group consisting of thermochromic liquid crystals, thermochromic leuco dyes, thermochromic oxides, optionally doped, and mixtures thereof. The thermochromic compound may be an optionally doped thermochromic oxide selected from the group consisting of VO₂, BiVO₄, NbO₂ and mixtures thereof. Tungsten-doped vanadium oxide VO₂:W is the preferred thermochromic compound. In particular, the tungsten doping rate can be chosen so that the tungsten-doped vanadium oxide VO₂:W has a transition temperature between 35° C. and 45° C., for example 40° C. Preferably, the tungsten doping rate of VO₂:W, defined as the ratio of tungsten mass to vanadium oxide mass, is between 0.5% and 1%. Vanadium oxide, doped or not, has a pseudo-rutile crystal form, called M, and is semiconducting below the transition temperature, and has a rutile crystal form, called R, and is metallic above the transition temperature. Forms M and R of vanadium oxide are different from forms A, B, C, M1 and M2, also known as vanadium oxide.

Vanadium oxide, doped or not, has an electrical resistivity contrast between the pseudo-rutile and rutile forms, which can be of the order of 10³ Ω·cm when the oxide structure is polycrystalline and of the order of 10⁴ Ω·cm when the oxide structure is single crystal. The variation in electrical resistivity results in a variation in the complex optical index, the optical properties of the oxide being modified by the release of charge carriers during phase change at the transition temperature.

As far as the optical filter is concerned, it may consist entirely of the thermochromic compound.

In particular, the optical filter is disposed so that the visible portion of the irradiation radiation transmitted by the optical filter and the light-collecting wall reaches, preferably directly, the culture chamber when the photobioreactor is exposed to the irradiation radiation. “Direct” access to the culture chamber means that the visible portion of the irradiation radiation transmitted by the combination of the light collection and the optical filter is not deflected before reaching the culture chamber.

Preferably, the optical filter is superimposed on the light-collecting wall and the culture chamber.

Preferably, the optical filter is in contact with the light-collecting wall. Preferably, it is in the form of a coating placed on one side of the light-collecting wall, preferably on the side of the light-collecting wall opposite the side of the light-collecting wall facing the culture chamber. The coating may partially cover the face of the light-collecting wall on which it is placed. For example, it can cover more than 30%, even more than 50%, or even more than 80% of the area of the side of the light-collecting wall on which it is placed. Preferably, it covers the entire side of the light-collecting wall on which it is placed.

The coating may have outer and inner surfaces, the inner surface being in contact with the light-collecting wall, the outer surface being opposite the inner surface. The outer and inner surfaces of the coating can be parallel.

The outer surface and/or the inner surface of the coating preferably have a rough texture in the form of a network formed by a regular succession of a relief pattern.

The relief pattern can have a variety of shapes, including pyramidal.

The rough texture of the outer surface and/or the rough texture of the inner surface of the coating limits the reflection of incident light radiation, particularly solar radiation, which has an impact that varies according to the time of day. The rough texture of the outer surface and/or the rough texture of the inner surface of the coating thus improves the optical transmittance of the optical filter to radiation in the incident visible, in particular to the visible component of solar radiation.

In the variant where the inner and outer surfaces of the coating each have a rough texture, the relief patterns on the outer and inner surfaces of the coating may be identical. Preferably, they are different, in order to maximize the anti-reflective function provided by each rough texture by taking into account the variation in optical index between the media that each of the outer and inner surfaces of the coating separates.

The rough texture of the outer surface of the coating preferably has a medium pitch, corresponding to the average distance between two consecutive reliefs of the relief pattern, between 50 nm and 300 nm, preferably between 50 nm and 100 nm, and/or an average height of the relief patterns between 100 nm and 600 nm, preferably between 100 nm and 200 nm. The average pitch and height of the patterns can be measured on photographs acquired using a scanning electron microscope.

The texturing ratio is equal to the ratio of the average height of the relief patterns to the average pitch. The texturing ratio of the outer surface is preferably between 1.3 and 2.0, preferably between 1.5 and 1.9.

The rough texture of the outer surface of the coating can be obtained by a process comprising a step of dry etching the side of the light-collecting wall on which the coating is placed, followed by a step of depositing the coating on said thus etched side. Different roughnesses of the rough texture of the outer surface of the coating can be obtained by varying the operating parameters of the dry etching step, which will be described below. The rough texture of the outer surface of the coating can thus define a replica of the texture of the side of the light-collecting wall on which the coating is placed.

The coating can be obtained by a deposition process selected from chemical vapor deposition and physical vapor deposition, especially magnetron sputtering.

The photobioreactor may include two optical filters, each in the form of a coating and disposed on the two opposite sides of the light-collecting wall.

The optical filter may have a support that is transparent to visible radiation and infrared radiation covered by the coating. The support is, for example, made of a material identical to the material of the light-collecting wall. According to this other variant, the optical filter is located at a distance from the enclosure, for example.

The optical filter may have an optical transmittance to infrared radiation greater than or equal to 80%, or even greater than or equal to 90%, respectively less than or equal to 20%, or even less than or equal to 10%, at a temperature at least 5° C. below, respectively at least 5° C. above the transition temperature of the thermochromic compound.

Preferably, the light-collecting wall comprises, for more than 90% of its mass, a material chosen from a glass, preferably a borosilicate glass or a soda-lime glass, and a thermoplastic polymer, preferably a polycarbonate or a polymethyl methacrylate.

The light-collecting wall may have an outer side facing the culture chamber and an inner side opposite the outer side and in contact with the optical filter.

The outer side and/or the inner side of the light-collecting wall may have a rough texture, preferably in the form of a network formed by a regular succession of a relief pattern. The rough texture of the outer side of the light-collecting wall limits the reflection of incident light radiation, particularly solar radiation, which has an impact that varies according to the time of day. It increases the optical transmittance of the light-collecting wall and the combination of the light-collecting wall and the optical filter to radiation in the incident visible, in particular to the visible component of solar radiation. The rough texture of the outer side of the light-collecting wall preferably has an average pitch between two consecutive reliefs of between 40 nm and 600 nm, preferably of between 80 nm and 300 nm, and/or an average height of the relief patterns of between 70 nm and 800 nm, preferably of between 100 nm and 600 nm.

The texturing ratio of the inner side of the light-collecting wall is preferably between 1.30 and 2.00, preferably between 1.50 and 1.90, in particular so as to ensure homogeneity of the thickness of the optical filter, when the latter is deposited, as described above, in the form of a coating on the inner side of the light-collecting wall.

The rough texture of the inner side of the light-collecting wall can be obtained by a dry etching process. In particular, it can be obtained by a vacuum plasma etching process, preferably operated by means of a gas mixture of trifluoromethane CHF₃ and dioxygen O₂, the molar ratio CHF₃/O₂ being between 10.0 and 15.0, under a pressure of between 50 mTorr and 200 mTorr, with a working density of between 1.65 W·cm⁻² and 3.56 W·cm⁻² and for a period of between 10 minutes and 30 minutes.

The presence of a rough texture improves the optical transmittance of the light-collecting wall.

In particular, the light-collecting wall may have a total reflection, expressed in percentages, of less than 2.5%, preferably less than 2.2%, preferably less than 1.7% or even less than 1.3%.

In addition, the light-collecting wall may have a haze ratio, defined as the ratio of diffuse reflection to total reflection and expressed in percentages, greater than 20%, preferably greater than 50%, or preferably greater than 80%.

The outer and inner sides of the light-collecting wall may have different rough textures.

In addition, in order to limit the formation of a biofilm by accumulation of the photosynthetic microorganism on the outer side of the light-collecting wall when the culture medium is in contact with said wall, the texturization ratio of the outer side of the light-collecting wall is preferably between 40 nm and 600 nm, preferably between 80 nm and 300 nm. In addition, the average pitch of the rough texture of the outer side of the light-collecting wall is preferably smaller than the size of the photosynthetic microorganism. The “size” of a synthetic microorganism is its largest dimension and can be measured by fluorimetry. The prevention of the formation of an opaque biofilm on the outer side of the light-collecting wall prevents the intensity of the radiation in the visible incident transmitted into the culture chamber from gradually decreasing from day to day during the culture of the photosynthetic microorganism.

The rough texture of the outer side of the light-collecting wall can be obtained by the vacuum plasma etching process described above.

The light-collecting wall may have the shape of a plate, particularly a cuboid shape.

Preferably, the optical filter is disposed so that the portion of the visible radiation transmitted by the optical filter and the light-collecting wall reaches the culture chamber, when the photobioreactor is exposed to visible radiation.

Preferably, the light-collecting wall is located between the culture chamber and the optical filter.

As far as the enclosure is concerned, it includes, or may even consist of, the light-collecting wall.

In a variant, the enclosure may have a tank, the light-collecting wall defining a tank cover. The tank can be made of the same material as the light-collecting wall.

The enclosure may have one or more openings configured so that the culture medium can flow through the culture chamber. In particular, it may include an inlet opening and an outlet opening for the culture medium.

The photobioreactor may also include a pump configured to circulate the culture medium through the culture chamber. In particular, the circulation can be in a closed loop, i.e. the culture medium discharged from the culture chamber through an outlet opening is reinjected into the culture chamber through one of the openings.

The enclosure can be of various shapes. In particular, it may have a general shape of a parallelepiped of revolution. It can be shaped like a hollow tube, which may be open at its ends. The tube generator can follow a helical curve. The tube may have straight portions and curved portions, for example curved at 90° or 180°, separating two consecutive straight portions, for example so that the tube winds in a zigzag pattern in a plane.

In addition, the enclosure defines the culture chamber. The culture chamber can contain the culture medium. The culture medium contains the photosynthetic microorganism and preferably contains an aqueous solvent containing nutrients in which the photosynthetic microorganism is dispersed.

The photosynthetic microorganism can be chosen from a photosynthetic bacterium, a photosynthetic cyanobacterium, a microalga. The preferred photosynthetic microorganism is a microalga, particularly Phaeodactylum tricornutum.

Preferably, the transition temperature of the thermochromic compound is lower than the temperature at which death of the photosynthetic microorganism is observed.

The invention also relates to a process for culturing a photosynthetic microorganism, in which a photobioreactor according to the invention is exposed to irradiation radiation comprising components in the visible and in the infrared, the culture chamber of the photobioreactor containing a culture medium comprising a photosynthetic microorganism.

The irradiation radiation can be solar radiation.

The culture medium, the photosynthetic microorganism, the visible radiation and the infrared radiation are preferably as described above.

### BRIEF DESCRIPTION OF THE FIGURES

Other aspects of the invention will appear more clearly when reading the detailed description below and the drawings in which:

FIG. 1 shows, schematically and in cross section, an example of a photobioreactor according to the invention;

FIG. 2 is an enlargement of a part of the photobioreactor of FIG. 1;

FIG. 3 a) to c) are photographs, taken by scanning electron microscopy, of the rough texture of the inner side of the light-collecting wall of different examples of photobioreactors according to the invention.

FIG. 4 illustrates the variation, as a function of the wavelength of the incident radiation, of the optical transmittance of the light-collecting walls shown of FIGS. 3a to 3c; and

FIG. 5 illustrates the variation, as a function of the wavelength of the incident radiation, of the haze ratio of the light-collecting walls shown in FIGS. 3a to 3c;

FIG. 6 illustrates the variation, as a function of the wavelength of the incident radiation, of the total reflection weighted by the spectral response of the eye of the light-collecting walls shown in FIGS. 3a to 3c; and

FIG. 7 illustrates another example of a photobioreactor according to the invention.

For reasons of clarity, the different elements of the figures are represented with a free scale, the actual dimensions of the different parts not necessarily being respected. In particular, the dimensions of the patterns of the rough textures may be exaggerated compared to the dimensions of the other components of the photobioreactor.

Hereinafter, the terms “between . . . and . . . ”, “from . . . to . . . ” and “varying from . . . to . . . ” are equivalent and mean that the boundaries are included, unless otherwise stated.

### DETAILED DESCRIPTION

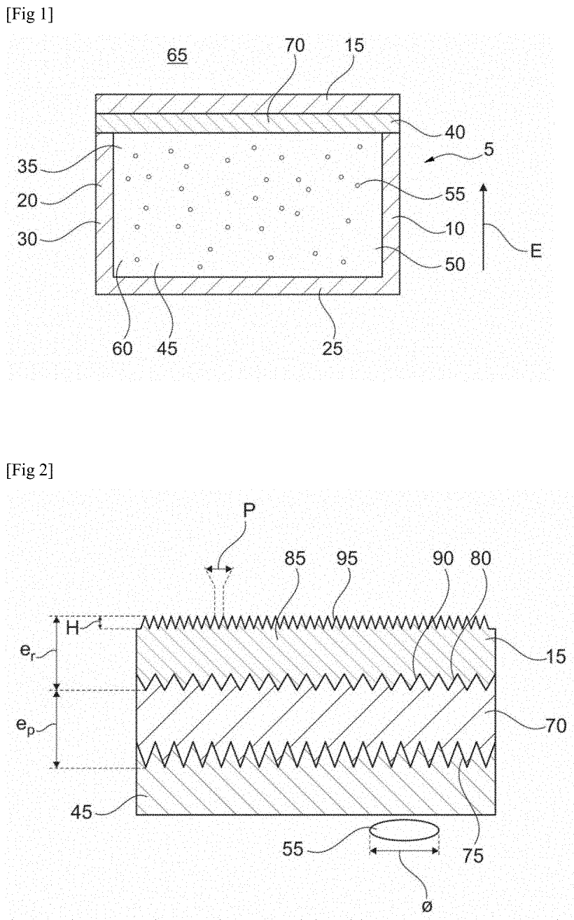

FIG. 1 shows an example of a photobioreactor 5 according to the invention. The photobioreactor has a chamber 10 and an optical filter 15 disposed in contact with the chamber.

The enclosure comprises a tank 20, with a bottom 25 and at least one side wall 30 extending from the bottom in a direction of extension E. The direction of extension may be vertical, although other orientations may be considered. The tank defines an upper tank opening 35.

The enclosure also has a cover 40, in the form of a plate, placed on the tank and completely covering the upper tank opening, so as to close the enclosure. The tank and cover thus define a culture chamber 45, which contains a culture medium 50 containing the photosynthetic microorganism 55. The culture medium contains an aqueous solvent 60 containing elements essential for the growth of the photosynthetic microorganism. The culture chamber can be hermetically sealed from the outside 65, for example by means of seals, not shown, sandwiched between the tank and the cover.

In the example of FIG. 1, the light-collecting wall 70 is defined by the cover 40. The light-collecting wall is transparent to sunlight and is made of glass, for example. The bottom and side walls of the tank can be made of a material that is opaque to visible radiation. In a variant, the bottom wall and the bottom side wall of the tank can be light-collecting walls.

The light-collecting wall has an outer side 75 facing the culture chamber and an inner side 80, opposite the outer side and separated by the thickness eₚ of the light-collecting wall. The optical filter 15 is in contact with and covers the inner side 80 of the light-collecting wall. In a variant, the optical filter can cover the outer side of the wall.

The optical filter is formed by a coating 85 made of a thermochromic material, for example tungsten-doped vanadium oxide, with a transition temperature between 38° C. and 42° C. The coating preferably has a thickness eᵣ between 50 nm and 800 nm. It has an inner surface 90 in contact with the inner side of the light-collecting wall and an outer surface 95 located opposite said inner surface.

In addition, as shown in FIG. 2, the outer surface 95 of the optical filter and the inner side 80 of the light-collecting wall each have a rough texture to increase the optical transmittance to visible radiation of the light-collecting wall and the optical filter. The rough texture is formed by the regular repetition of a pattern of triangular cross section. The texture has a pitch P between two patterns and a pattern height H.

In addition, the outer side 75 of the light-collecting wall has a rough texture, which is also formed by the regular repetition of a pattern such as the outer surface of the optical filter. However, the average pitch of the rough texture of the outer side of the light-collecting wall is less than the size ϕ of the microorganism, in order to limit the formation of an opaque biofilm comprising the photosynthetic microorganism on the light-collecting wall.

The photobioreactor may also include means, not shown, for renewing the aqueous solvent and/or supplying nutrients to the culture medium, such as a source of potassium or a gas such as carbon dioxide. It may include means for extracting the products of the growth of the photosynthetic microorganism, for example a gas such as dioxygen generated by photosynthetic activity.

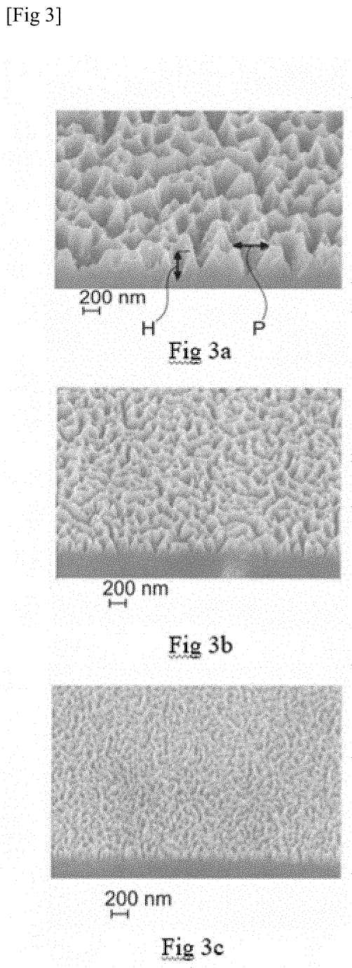

FIGS. 3a to 3c are scanning microscopy photographs of the rough texture of the inner side of the light-collecting wall of three photobioreactors, hereinafter referred to as Examples 1 to 3. The light-collecting wall shown in these figures is an aluminoborosilicate glass plate.

The rough textures shown in FIGS. 3a to 3c were obtained by vacuum plasma treatment of the light-collecting wall using dry etching equipment. The dry etching process was carried out using a gas mixture of trifluoromethane CHF₃ and dioxygen O₂ with a CHF₃/O₂ ratio between 10 and 15, at a working pressure between 50 mTorr and 200 mTorr, with a power density between 1.65 W/cm² and 3.56 W/cm² (RF), and during a treatment time between 10 minutes and 30 minutes.

As can be seen in FIGS. 3a to 3c, the rough texture has a substantially regular structure formed by the repetition, depending on the width and length of the coating, of a pattern with a pyramidal shape.

The variation in working pressures, power density and processing time results in variation in the average pitch and height of the texture.

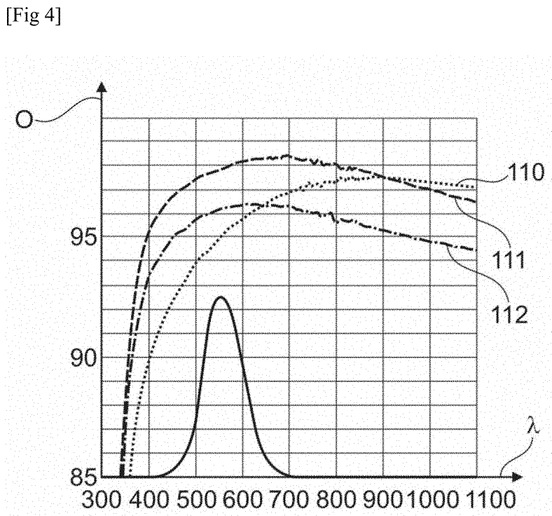

FIG. 4 illustrates the changes 110, 111 and 112 as a function of the wavelength λ, expressed in nm, of the optical transmittance, expressed in percent, of the light-collecting walls of Examples 1 to 3, respectively. The collecting walls of Examples 1 to 3 are transparent to visible and infrared radiation with a wavelength of less than 1100 nm.

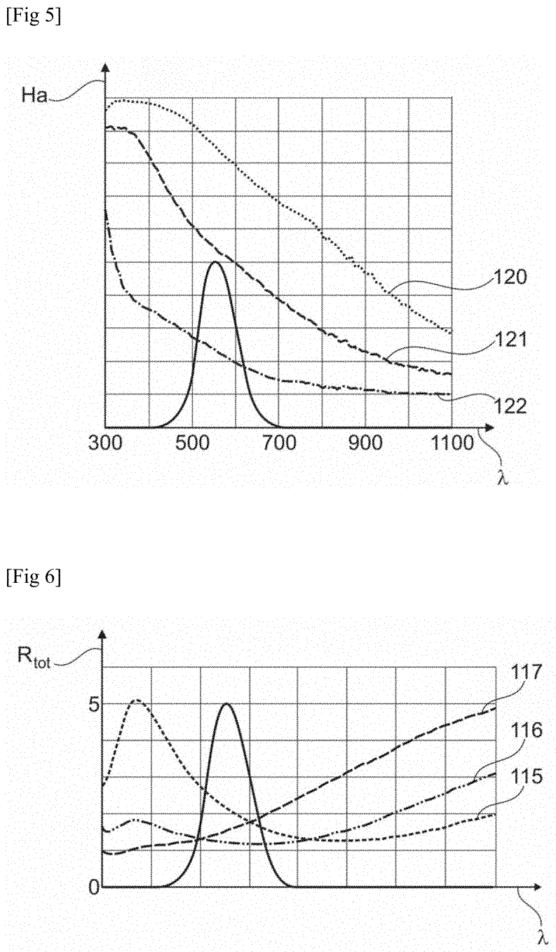

FIG. 5 illustrates the changes 115, 116 and 117 as a function of the wavelength λ, expressed in nm, of the total reflection Rₜₒₜ, expressed in percent, of the light-collecting walls of Examples 1 to 3, respectively. The presence of a rough texture on the inner side of the collecting walls of Examples 1 to 3, although not essential to the invention, limits the total reflection of incident radiation. The total reflection, shown in FIG. 5, is always less than 6% regardless of the wavelength of the incident radiation between 350 nm and 1100 nm for Examples 1 to 3. The total response weighted by the spectral response of the eye is at most 2.16% for Example 1, as shown in Table 1. It is of the order of 8% for a collecting wall formed from the same material but not having a rough texture.

FIG. 5 illustrates the changes 120, 121 and 122 as a function of the wavelength λ, expressed in nm, of the haze ratio Ha of the light-collecting walls of Examples 1 to 3, respectively. Weighted by the spectral response of the eye, the haze ratio is at least 22.0% (Example 3). The collecting wall of the example not treated with plasma, not having a rough texture, has a haze ratio of less than 1.0%.

The presence of a rough texture, although not essential to the invention, therefore improves the optical transmittance of the assembly formed by the optical filter and the light-collecting wall.

TABLE 1

| | | | | Without plasma |
|---|---|---|---|---|
| Example | 1 | 2 | 3 | treatment |
| Total reflection (TR) | 2.16 | 1.23 | 1.59 | 8 |
| weighted by the spectral | | | | |
| response of the eye (%) | | | | |
| Minimum total reflection | 1.28 | 1.16 | 1.11 | 8 |
| over the wavelength range | | | | |
| 400 nm to 800 nm (%) | | | | |
| Wavelength for which the | 785 | 610 | 330 | Not applicable |
| total reflection is minimal | | | | |
| (nm) | | | | |
| Diffuse reflection (DR) | 1.84 | 0.66 | 0.35 | <0.5 |
| weighted by the spectral | | | | |
| response of the eye (%) | | | | |
| Haze ratio = DR/TR (%) | 85.2 | 53.7 | 22.0 | <1.0 |

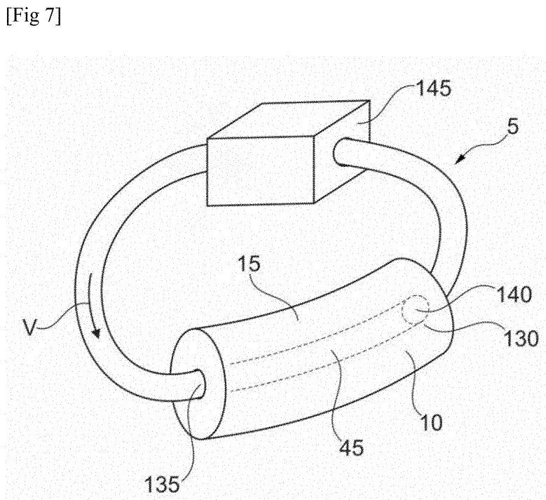

FIG. 7 illustrates another example of a photobioreactor according to the invention.

The photobioreactor 5 of FIG. 7 differs from the photobioreactor shown in FIG. 1 in that the enclosure 10 consists of the light-collecting wall, the outer side of which 130 is defined by the outer surface side of the tube, and is covered by the optical filter 15 as a coating formed of the thermochromic compound. The enclosure has a general tubular shape of revolution and has inlet openings 135 and outlet openings 140 through which the culture medium 45 flows into and out of the enclosure, respectively. The circulation V of the culture medium is carried out in a closed loop. A pump 145 draws the culture medium leaving the chamber through the outlet opening 140 and reinjects it through the inlet opening 135. As is apparent from the reading the description, the photobioreactor according to the invention ensures a passive regulation of the temperature of the culture medium, thus promoting culture, at low cost with a high yield of the photosynthetic microorganism.

Of course, the invention is not limited to the exemplary embodiments of the device and of implementation of the process described above.

1. Photobioreactor for culturing a photosynthetic microorganism, the photobioreactor comprising:
an enclosure having a light-collecting wall and defining a culture chamber for containing a culture medium containing at least the photosynthetic microorganism, the light-collecting wall being transparent to infrared and visible radiation, and
an optical filter for filtering radiation directed toward the culture chamber,
the optical filter being transparent to radiation in the visible and containing at least one thermochromic compound,
the optical filter being transparent to infrared radiation at a temperature at least 10° C. below the transition temperature of the thermochromic compound and having an optical transmittance to infrared radiation of 20% or less at a temperature at least 10° C. above the transition temperature of the thermochromic compound.

2. Photobioreactor according to claim 1, wherein the transition temperature is greater than or equal to 30° C.

3. Photobioreactor according to claim 1, wherein the thermochromic compound is an optionally doped thermochromic oxide selected from the group consisting of VO₂, BiVO₄, NbO₂ and mixtures thereof.

4. Photobioreactor according to claim 1, wherein the optical filter has an optical transmittance to infrared radiation greater than or equal to 80%, or even greater than or equal to 90%, respectively less than or equal to 20%, or even less than or equal to 10%, at a temperature at least 5° C. below, respectively at least 5° C. above the transition temperature of the thermochromic compound.

5. Photobioreactor according to claim 1, wherein the optical filter is disposed on the light-collecting wall.

6. Photobioreactor according to claim 1, wherein the optical fitter is formed by a coating covering at least partially or completely one side of the light-collecting wall.

7. Photobioreactor according to claim 6, wherein the coating has outer and inner surfaces, the inner surface being in contact with the light-collecting wall, the outer surface being opposite the inner surface and having a rough texture in the form of a network formed by a regular succession of a relief pattern.

8. Photobioreactor according to claim 1, wherein the optical filter is disposed so that the visible portion of the irradiation radiation transmitted by the optical filter and by the light-collecting wall reaches the culture chamber, when the photobioreactor is exposed to visible radiation.

9. Photobioreactor according to claim 1, wherein the light-collecting wall is disposed between the culture chamber and the optical filter.

10. Photobioreactor according to claim 1, wherein the light-collecting wall has an outer side facing the culture chamber and an inner side opposite the outer side and in contact with the optical filter, the outer side or the inner side of the light-collecting wall having a rough texture.

11. Photobioreactor according to claim 1, wherein the texturing ratio of the inner side of the light-collecting wall is between 1.30 and 2.00.

12. Photobioreactor according to claim 1, wherein the culture chamber contains the culture medium, the photosynthetic microorganism being selected from a photosynthetic bacterium, a photosynthetic cyanobacterfum, and an especially eukaryotic microalga.

13. Photobioreactor according to claim 1, wherein the outer side of the light-collecting wall has a rough texture whose average pitch is less than the size of the photosynthetic microorganism.

14. Process for culturing a photosynthetic microorganism, wherein a photobioreactor according to claim 1 is exposed to irradiation radiation comprising components in the visible and infrared, the culture chamber of the photobioreactor containing a culture medium comprising a photosynthetic microorganism.

15. Process according to claim 14, wherein the irradiation radiation is solar radiation.

16. Photobioreactor according to claim 1, wherein the transition temperature is greater than or equal to 35° C. or less than or equal to 55° C.

17. Photobioreactor according to claim 1, wherein the transition temperature is less than or equal to 50° C.

18. Photobioreactor according to claim 1, wherein the transition temperature is less than or equal to 45° C.

19. Photobioreactor according to claim 3, wherein the thermochromic compound is tungsten-doped vanadium oxide VO₂:W.

20. Photobioreactor according to claim 10, wherein the outer side or the inner side of the light-collecting wall has a rough texture in the form of a network formed by a regular succession of a relief pattern.

21. Photobioreactor according to claim 11, wherein the texturing ratio of the inner side of the light-collecting wall is between 1.50 and 1.90.
